(19) United States
(12) Patent Application Publication
(10) Pub. No.: US 2026/0338307 A1
(43) Pub. Date: Oct. 29, 2026

(54) DUAL-PORT CONNECTOR FOR RECEPTACLE OF SURGICAL CONSOLE

(71) Applicant: Alcon Inc., Fribourg (CH)

(72) Inventor: Nathaniel Reyes, Irvine, CA (US)

(21) Appl. No.: 19/574,653

(22) Filed: Mar. 23, 2026

Related U.S. Application Data
(60) Provisional application No. 63/793,348, filed on Apr. 23, 2025.

(51) Int. Cl. F16L 39/02; A61F 9/007; A61B 17/00

(52) U.S. Cl. CPC F16L 39/02; A61F 9/007; A61B 2017/00199

(57) ABSTRACT

The present disclosure generally relates to implementations of a dual-port connector that connects to a receptacle of a surgical console. The dual-port connector includes a core member having first and second connector channels that fluidly connect receptacle channels with first and second tubes connected to a surgical instrument, and one or more spring arms that retain the dual-port connector in the receptacle. The dual-port connector further includes a lid member disposed around the core member. The lid member includes one or more lateral openings through which a portion of the one or spring arms extends to connect the core member to the receptacle, and one or more release features that depress the one or more spring arms to release the dual-port connector from the receptacle.

Vitreo-retinal procedures may include a variety of surgical procedures performed to restore, preserve, and enhance vision. Vitreo-retinal procedures may be appropriate to treat many serious conditions of the back of the eye. For example, vitreo-retinal procedures may treat conditions such as age-related macular degeneration (AMD), diabetic retinopathy and diabetic vitreous hemorrhage, macular hole, retinal detachment, epiretinal membrane, cytomegalovirus (CMV) retinitis, and many other ophthalmic conditions.

The vitreous is a normally clear, gel-like substance that fills the center of the eye. The vitreous may constitute approximately two-thirds of the eye's volume, giving it form and shape before birth. Certain problems affecting the back of the eye may require a vitrectomy, or a surgical removal of the vitreous. Removal of vitreous can involve a vitrector (sometimes referred to as a “cutter” or a “vitreous cutter”) that operates like a tiny guillotine, having an oscillating microscopic cutter to remove the vitreous gel in a controlled fashion. The cutter is powered by a pneumatic vitrectomy machine (sometimes implemented as a surgical console) including one or more pneumatic valves (sometimes referred to as “drive valves”). For example, the cutter may be powered by pressurized gas that is alternately directed to two output ports of the surgical console. In certain cases, the output ports are coupled to the cutter through two separate tubes, each having a separate port connector that attaches to one of the output ports.

In certain cases, there may be only a limited amount of area available on the surgical console for port connectors. As such, providing space for two port connectors in such a limited space may require that each port connector is configured to have a narrower air channel, resulting in less air flow through each port connector. In addition, coupling two port connectors to the surgical console may be cumbersome.

### SUMMARY

The present disclosure generally relates to implementations of a dual-port connector suitable for providing multiple fluid connections between a surgical instrument and a receptacle of a surgical console. In some embodiments, the receptacle of the surgical console has multiple output ports that deliver pressurized gas to a vitrector by two separate tubes that attach to the dual-port connector. Although vitreous surgery is discussed as an example of a surgical procedure that may benefit from the described embodiments, the advantages may benefit other types of surgical instruments and procedures as well.

In some embodiments, a dual-port connector is configured to connect to a receptacle of a surgical console. The dual-port connector comprises a core member comprising: a first connector channel configured to fluidly connect a first receptacle channel of the receptacle with a first tube connected to a surgical instrument; a second connector channel configured to fluidly connect a second receptacle channel of the receptacle with a second tube connected to the surgical instrument; and one or more spring arms configured to retain the dual-port connector in the receptacle. The dual-port connector further comprises a lid member disposed around the core member, the lid member comprising: one or more lateral openings through which a portion of the one or more spring arms extends to connect the core member to the receptacle; and one or more release features configured to depress the one or more spring arms to release the dual-port connector from the receptacle.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope and may admit to other equally effective embodiments.

FIG. 1A illustrates an embodiment of a surgical console for a pneumatically-powered ophthalmic surgical machine, in accordance with certain embodiments.

FIG. 1B illustrates a block diagram of the surgical console of FIG. 1A, according to one or more embodiments.

FIG. 2 illustrates a perspective view of an interface component of a dual-port receptacle, according to one or more embodiments.

FIG. 3A illustrates a perspective view of an embodiment of a dual-port connector, according to one or more embodiments.

FIG. 3B illustrates a perspective view of an embodiment of a dual-port connector, according to one or more embodiments.

FIG. 4 illustrates a perspective view of a partial assembly of the dual-port connector, according to one or more embodiments.

FIG. 5 illustrates a perspective view of a sleeve member of the dual-port connector, according to one or more embodiments.

FIG. 6A illustrates a cross-sectional view of the dual-port connector in a retained configuration in the receptacle, according to one or more embodiments.

FIG. 6B illustrates a cross-sectional view of the dual-port connector when releasing from the receptacle, according to one or more embodiments.

FIG. 6C illustrates an alternate cross-sectional view of the dual-port connector in a retained configuration in the receptacle, according to one or more embodiments.

FIG. 7 illustrates a cross-sectional view of the core member having a first tube and a second tube retained therein, according to one or more embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with various other embodiments discussed herein. In similar fashion, while exemplary embodiments may be discussed below as device, instrument, or method embodiments it should be understood that such exemplary embodiments can be implemented in various devices, instruments, and methods.

Embodiments of the present disclosure are directed to implementations of a dual-port connector capable of supporting multiple fluid connections between a surgical instrument and a receptacle of a surgical console. For example, the dual-port connector may be used to transmit air pressures from a surgical console to operate a vitrectomy probe. In some embodiments, the dual-port connector is implemented as a snap-fit connector that fluidly connects tubes with corresponding channels of a receptacle. The snap-fit connector comprises a core having one or more spring arms, and a sleeve disposed around the core. The one or more spring arms extend through one or more lateral openings of the sleeve into one or more recesses of the receptacle to retain the snap-fit connector in the receptacle.

The sleeve further includes one or more release features that, responsive to an axial pulling force applied to the lid (or to a sleeve connected thereto), depress the one or more spring arms to release them from the one or more recesses, which allows the snap-fit connector to be withdrawn from the receptacle. In some embodiments, applying the axial pulling force to the core (or to the tubing connected thereto) does not release the snap-fit connector from the receptacle. In this way, the dual-port connector supports a simple connection and disconnection from the surgical console by a surgeon or technician (e.g., pushing and pulling of the sleeve), while providing a robust connection between the snap-fit connector and the receptacle that can resist separation due to pressurization of air flow paths and withstand incidental forces (e.g., an unintentional pulling of the tubes) experienced during a surgical procedure.

In some embodiments, various components of the snap-fit connector (e.g., the core, the sleeve, the lid) may themselves be snap-fit together to assemble the snap-fit connector. Consequently, the components of the snap-fit connector can be manufactured with tighter clearances than when joined using techniques such as ultrasonic welding, which requires certain clearances to ensure that components are not inadvertently welded together. Specifically, in some cases the sleeve is slidably connected to the core, which would require significant clearances to prevent inadvertent welding of the sleeve to the core, and to preserve the slidable connection. These greater clearances indicate that the components are not so tightly constrained, which makes the components more susceptible to misalignment and/or jamming during use. Using the snap-fit approach to construct the snap-fit connector thus results in improved performance, improved reliability, and/or a less complex manufacture (e.g., reduced cost, reduced waste) of the snap-fit connector. The snap-fit connector may further experience reduced internal stresses during use, when compared with an ultrasonic-welded connector, providing a larger margin to the yield strength of the material.

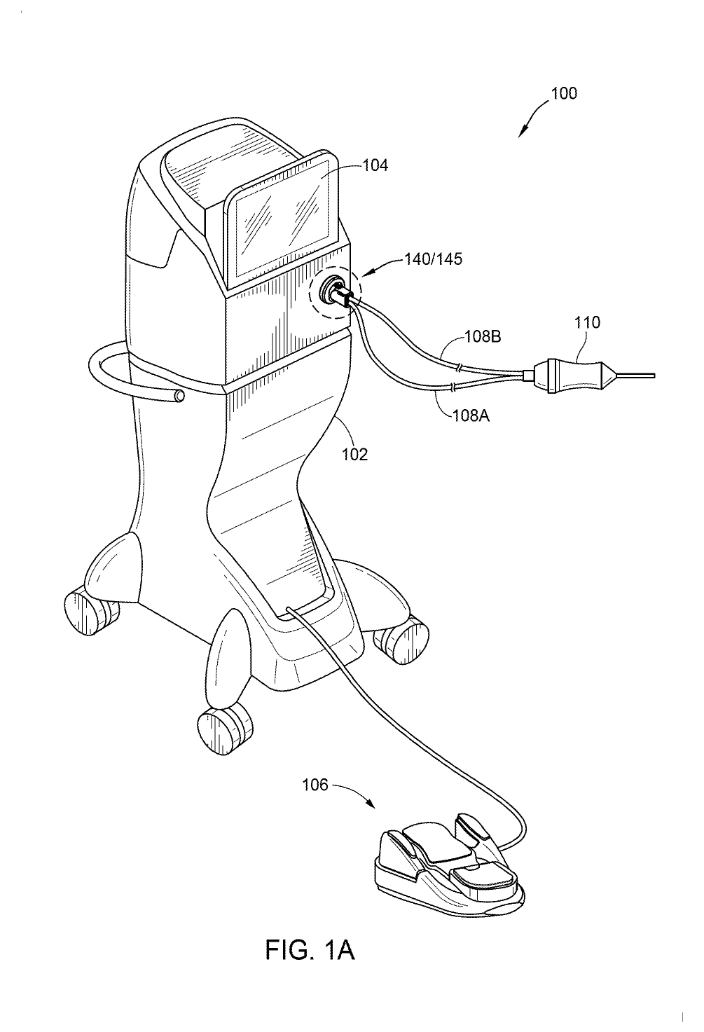

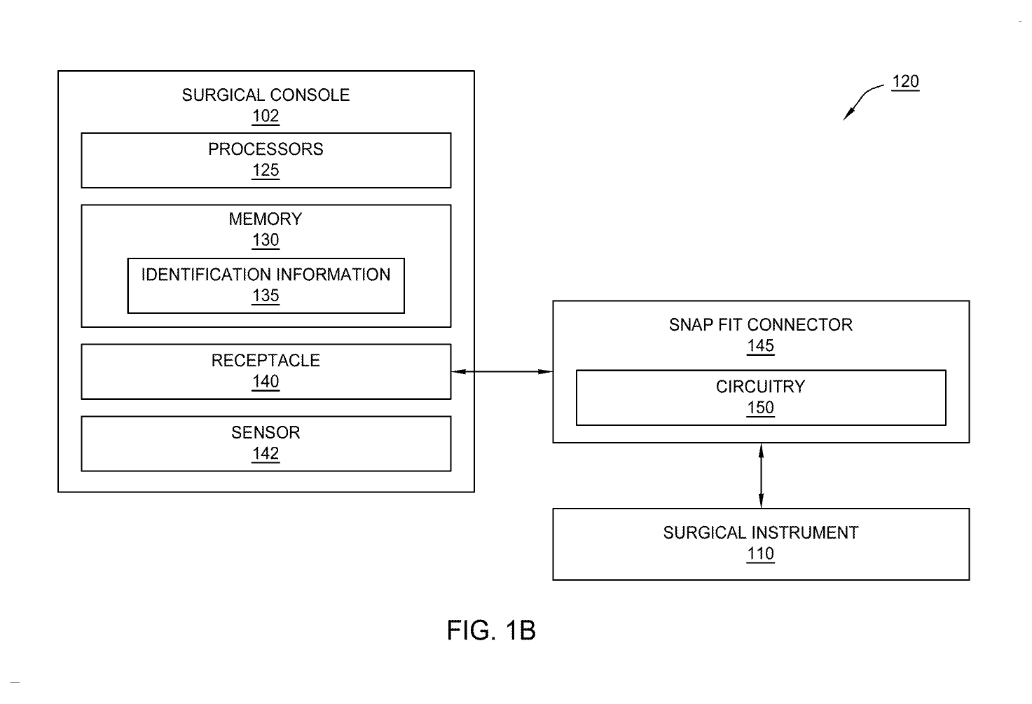

FIG. 1A illustrates an exemplary surgical console 102 (or “console 102”) of a surgical system 100, according to one or more embodiments. The surgical system 100 includes the surgical console 102 operatively connected with a surgical instrument 110, such as a vitrectomy probe handpiece. In some embodiments, the console 102 may be mobile, and may include wheels or casters that facilitate movement of the console 102 throughout an operating room. In some other embodiments, the console 102 may be statically positioned. The console 102 may be referred to as a “base housing” and may include a plurality of subsystems whose coordinated operation enables a surgeon to perform a variety of surgical procedures, such as ophthalmic surgical procedures.

The surgical instrument 110 attaches to the surgical console 102 by one or more connection conduits 108A, 108B of the surgical system 100. Although two connection conduits 108A, 108B are shown, different numbers are also contemplated (e.g., one, three, four or more). The connection conduits 108A, 108B may be operated to supply high and low fluid pressures, electrical power, optical power, control signals, and so forth to the surgical instrument 110. In some implementations, one or both of the connection conduits 108A, 108B are formed of lengths of tubing forming fluid conduits that convey fluids, such as air, saline, removed material (e.g., vitreous), substance(s), or others, between the surgical instrument 110 and one or more subsystems of the console 102. In some embodiments, the connection conduits 108A and/or 108B may each include a plurality of lumens that enable multiple fluids to be conveyed between the surgical instrument 110 and the console 102. Thus, in some embodiments, the surgical instrument 110 forms part of a vitrectomy subsystem described herein.

In certain cases, the surgical console 102 may have only a limited amount of area to arrange port connectors for connecting to each of the connection conduits 108A, 108B. Such limitations can require that the port connectors be implemented with narrower channels, reducing fluid flow capability and the overall functionality of the surgical instrument 110. In some embodiments, the surgical console 102 comprises a receptacle 140 (also referred to as a “dual-port receptacle”) that defines a first receptacle channel, a second receptacle channel, and one or more recesses. A connector 145 (also referred to as a “dual-port connector” or “snap-fit connector”) connects to the receptacle 140 and to the surgical instrument 110 through each of the connection conduits 108A, 108B (e.g., a first tube and a second tube).

In some embodiments, the connector 145 comprises a multi-part assembly including a core, a lid, and a sleeve. In one example implementation, the core is connected (e.g., physically attached) to the connection conduits 108A, 108B. The lid is attached to the sleeve, and the lid and the sleeve are each disposed around the core. The core defines one or more spring arms having portion(s) that extend through lateral openings of the lid, and that retain the connector 145 in the receptacle 140.

Continuing the example, the lid is slidably connected to the core and includes one or more release features that depress the one or more spring arms responsive to an axial pulling force on the lid (in some cases, transmitted to the lid through the attached sleeve). Applying an axial pulling force to the core (e.g., pulling on the attached tubing) does not release the connector 145 from the receptacle 140. Further description of the receptacle 140 and the connector 145 is provided below with respect to FIGS. 1B-7. In another example implementation of the connector 145, the lid and the sleeve may be formed as a single component and do not move relative to each other.

The console 102 may include an associated display 104 that is configured to visually display information relating to operation and performance of the surgical system 100 during a surgical procedure (e.g., a vitrectomy procedure). Although not shown, the console 102 may include other feedback devices (e.g., audio speakers, haptic motors) that provide information during the surgical procedure.

In some embodiments, the surgical system 100 further includes an input device 106 (also referred to as an “electronic accessory”) that may be any device that is capable of receiving commands from the user of the console 102 to operate the surgical instrument 110 and/or to provide control to other components of the surgical console 102. In FIG. 1, the input device 106 is illustrated as a foot controller, however, other types of input devices are also within the scope of the disclosure, such as a touchscreen integrated with the display 104, physical buttons included on the surgical console 102, and so forth. In one example, the user provides a command to the input device 106 (e.g., a surgeon using his or her foot to manipulate a pedal of the foot controller), which is transmitted to a controller of the console 102 (e.g., by an input subsystem of the console 102). In response, the controller transmits instructions to enable and/or control the operation of the surgical instrument 110 (e.g., by a handpiece subsystem and/or aspiration subsystem of the console 102) based on the user command. In some embodiments, the input device 106 further includes one or more feedback devices, such as a visual display, audio speakers, and/or haptic motors that provide information during a surgical procedure.

FIG. 1B illustrates a block diagram 120 of the surgical console 102 of FIG. 1, according to one or more embodiments. The features illustrated in FIG. 1B may be used in conjunction with other embodiments.

The surgical console 102 comprises one or more processors 125, a memory 130, the receptacle 140, and a sensor 142. The one or more processors 125 may include any type(s) of general-purpose processor and/or processor specifically designed for performing various functions described herein, such as an application-specific integrated circuit (“ASIC”). The one or more processors 125 may be, or may include, a microprocessor, a microcontroller, an embedded microcontroller, a programmable digital signal processor, or any other programmable device operable to execute instructions stored in the memory 130 for operating the surgical console 102, or combinations thereof. In some instances, the one or more processors 125 may also be, or may include, a programmable gate array, programmable array logic, or any other device of combinations of devices operable to process electronic signals.

The memory 130 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 130 stores instructions executed by the one or more processors 125. In example embodiments, functionality disclosed herein can be provided by the one or more processors 125 and the memory 130 (i.e., software-based), by other circuitry of the one or more processors 125 (i.e., hardware-based), or by a combination thereof. The memory 130 may include code stored thereon. The code may include instructions that may be executable by the one or more processors 125. The code may be created, for example, using any programming language, including but not limited to, C, C++, Java, Python, Rust, or any other programming language (including assembly languages, hardware description languages, and database programming languages). In some embodiments, the code may be a program that, when executed by the one or more processors 125, controls cutting and aspiration functions of the surgical instrument 110, provides real-time feedback to the surgeon, and so forth. In some embodiments, the memory 130 stores identification information 135 that identifies one or both of the snap-fit connector 145 and the surgical instrument 110, and the program (or other code) configures operation of the surgical console 102 responsive to the identification information 135. For example, the identification information 135 may include manufacturer information, model information, physical parameters (e.g., diameter of the tubing), and/or operational parameters.

In some embodiments, the sensor 142 is communicatively connected with the one or more processors 125 and detects the presence of the snap-fit connector 145 (in some cases, detecting the presence of the core of the connector 145) when inserted and retained in the receptacle 140. The sensor 142 may have any suitable implementation, e.g., a proximity sensor using any suitable sensing technology. In some embodiments, the one or more processors 125 (executes code that) initiates, responsive to the sensor 142 detecting the presence of the core, wireless communication with circuitry 150 of the snap-fit connector 145. In some embodiments, the circuitry 150 comprises Radio Frequency Identification (RFID) circuitry that provides the identification information 135 to the surgical console 102.

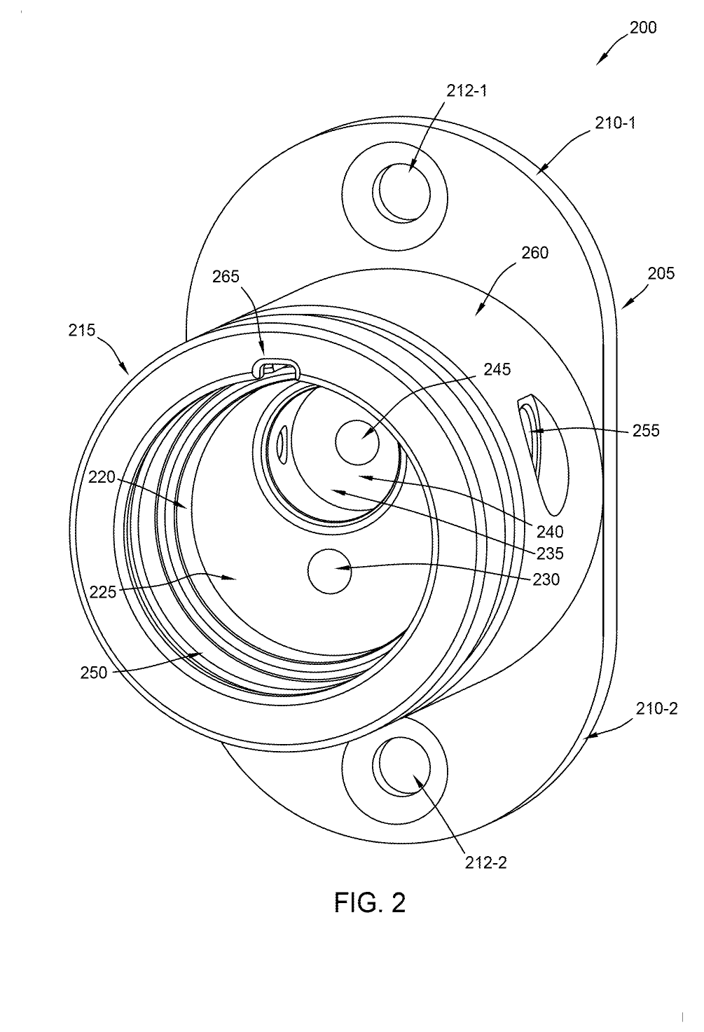

FIG. 2 illustrates a perspective view of an interface component 200 of a dual-port receptacle, according to one or more embodiments. The features illustrated in FIG. 2 may be used in conjunction with other embodiments. For example, the interface component 200 represents one example implementation of the receptacle 140.

The interface component 200 comprises a base 205 and a projecting section 215. The base 205 defines a first flange 210-1 and a second flange 210-2 disposed apart from each other, on opposing sides of the projecting section 215. The first flange 210-1 and the second flange 210-2 define respective fastener openings 212-1, 212-2 that may be used to attach the interface component 200 to a structure of the surgical console 102.

The projecting section 215 extends perpendicular to a plane of the base 205. The projecting section 215 has a substantially cylindrical shape, although other shapes are also contemplated. A recess 220 extends into the projecting section 215 from a distal surface. Note that, as described herein, a distal end or portion of a component refers to the end or the portion that is closer to a patient’s body during use thereof. On the other hand, a proximal end or portion of the component refers to the end or the portion that is distanced further away from the patient’s body. As shown, the recess 220 extends to a first recessed surface 225 at a first depth from the distal end and extends from the first recessed surface 225 to a second recessed surface 240 at a second depth from the distal end. Having the first recessed surface 225 and the second recessed surface 240 at different depths may be advantageous to ensure a desired orientation of the connector 145.

In some embodiments, the first recessed surface 225 and the second recessed surface 240 have circular shapes, although other shapes (and combinations of shapes) are also contemplated. A first receptacle channel 230 extends through the projecting section 215 to the first recessed surface 225, and a second receptacle channel 245 extends through the projecting section 215 to the second recessed surface 240.

In some embodiments, the connector 145 defines a first proximal male coupling and a second proximal male coupling that are received in the recess 220. The first proximal male coupling is received in the recess 220 and seats against the first recessed surface 225 to fluidly connect to the first receptacle channel 230, and the second proximal male coupling is received into a further recessed portion 235 and seats against the second recessed surface 240 to fluidly connect to the second receptacle channel 245.

The projecting section 215 further defines one or more internal circumferential channels 250 that receive portions of the connector 145 (e.g., portions of one or more spring arms) to retain the connector 145 in the receptacle 140. Although a single internal circumferential channel 250 is shown that extends around the entire circumference of the recess 220, in other embodiments the internal circumferential channel(s) 250 may extend around segment(s) of the circumference of the recess 220. Retaining the connector 145 using the segment(s) of the circumference may be advantageous to ensure a desired orientation of the connector 145.

In some embodiments, a lateral recess 255 is formed into the projecting section 215 from a lateral surface 260. The lateral recess 255 is operably connected to the recess 220 (in some cases, the further recessed portion 235) to enable the sensor 142 to detect the presence of the connector 145. In some embodiments, the sensor 142 may be disposed in the lateral recess 255. In other embodiments, the sensor 142 is disposed external to the projecting section 215 and senses through the lateral recess 255 (e.g., through free space, an optically transmissive material, a magnetically permeable material, an electrically permittive material, and so forth). In some embodiments, the sensor 142 is positioned to detect whether the connector 145 is fully seated in the receptacle 140 (e.g., the one or more spring arms have been received in the internal circumferential channel(s) 250). In some embodiments, the one or more spring arms may provide audible feedback (e.g., a click) that confirms to a user that the connector 145 is fully seated in the receptacle 140.

In some embodiments, a female keying feature 265 is formed in the projecting section 215 along an axial direction to receive a corresponding male keying feature of the connector 145 and ensure a desired orientation of the connector 145.

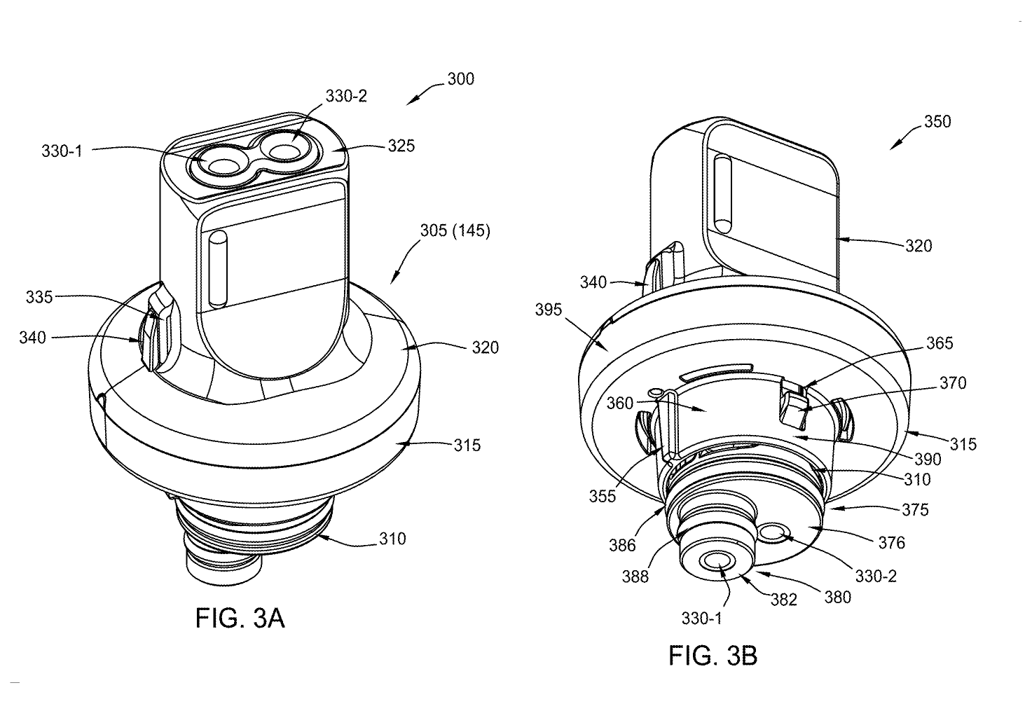

FIGS. 3A and 3B illustrate perspective views 300, 350 of a dual-port connector 305, according to one or more embodiments. The features illustrated in FIGS. 3A and 3B may be used in conjunction with other embodiments. For example, the dual-port connector 305 represents one example implementation of the connector 145.

The dual-port connector 305 comprises a core member 310, a lid member 315, and a sleeve member 320, each of which may be formed using any suitable materials such as polycarbonate, ABS (acrylonitrile butadiene styrene), nylon, copolyester, polystyrene, or other polymers. In some embodiments, each of the core member 310, the lid member 315, and the sleeve member 320 is implemented as a respective single component. The core member 310 extends along an axial direction and defines a first connector channel 330-1 and a second connector channel 330-2 that are arranged in parallel along the axial direction. The first connector channel 330-1 extends from a distal end 325 of the dual-port connector 305 to a proximal surface 376 of the core member 310 and the second connector channel 330-2 extends from the distal end 325 to a proximal surface 382 of the core member 310.

The core member 310 defines a first proximal male coupling 375 that extends beyond the lid member 315, and a second proximal male coupling 380 that extends beyond the first proximal male coupling 375. In some embodiments, the first proximal male coupling 375 defines the proximal surface 376, which is received in the recess 220 and seats against the first recessed surface 225 to fluidly connect the first connector channel 330-1 with the first receptacle channel 230. The second proximal male coupling 380 defines the proximal surface 382, which is received in the further recessed portion 235 and seats against the second recessed surface 240 to fluidly connect the second connector channel 330-2 with the second receptacle channel 245. In some embodiments, the first proximal male coupling 375 includes a circumferential groove 386 that receives a compliant washer to form a sealing surface in the recess 220, and the second proximal male coupling 380 includes a circumferential groove 388 that receives an O-ring of a compliant material to form a sealing surface in the further recessed portion 235.

The lid member 315 is disposed around the core member 310. As shown, the lid member 315 defines a base portion 395 and an annular portion 360 that extends proximally from the base portion 395. The base portion 395 connects to the sleeve member 320, e.g., rigidly attaching to each other using complementary retaining features. For example, the sleeve member 320 defines a lateral opening 335 through which a protrusion 340 of the lid member 315 extends when the sleeve member 320 is seated against the base portion 395. Having the lateral opening 335 and the protrusion 340 may be advantageous to ensure a desired orientation of the lid member 315 relative to the sleeve member 320.

In some embodiments, a diameter of the sleeve member 320 at the proximal end is approximately the same as the diameter of the distal end of the lid member 315, and the sleeve member 320 is contoured with a decreasing diameter towards the distal end 325 of the dual-port connector 305. In some embodiments, the sleeve member 320 may be knurled or textured along the external surface to promote gripping by a surgeon or technician.

The core member 310 extends through the sleeve member 320, the base portion 395, and the annular portion 360, extending beyond the annular portion 360 to define the first proximal male coupling 375 and the second proximal male coupling 380. The annular portion 360 defines one or more lateral openings 365 through which a portion of one or more spring arms 370 of the core member 310 extends. These extended portions(s) of the spring arm(s) 370 connect the core member 310 to the receptacle 140 (e.g., retained in the internal circumferential channel(s) 250) to retain the dual-port connector 305 in the receptacle 140.

In some embodiments, the lid member 315 further defines a male keying feature 355 extending along the axial direction that is received in the female keying feature 265 of the receptacle 140.

The lid member 315 further defines one or more release features 390 that depress the one or more spring arms 370 to release the dual-port connector 305 from the receptacle 140. In some embodiments, the one or more release features 390 comprise proximal sections of the annular portion 360. In some embodiments, the lid member 315 is slidably connected to the core member 310, such that an axial pulling force on the lid member 315 causes the lid member 315 to slide distally, which causes the one or more release features 390 to depress the portion of the one or more spring arms 370 (e.g., to apply a medial force) and release the dual-port connector 305 from the receptacle 140.

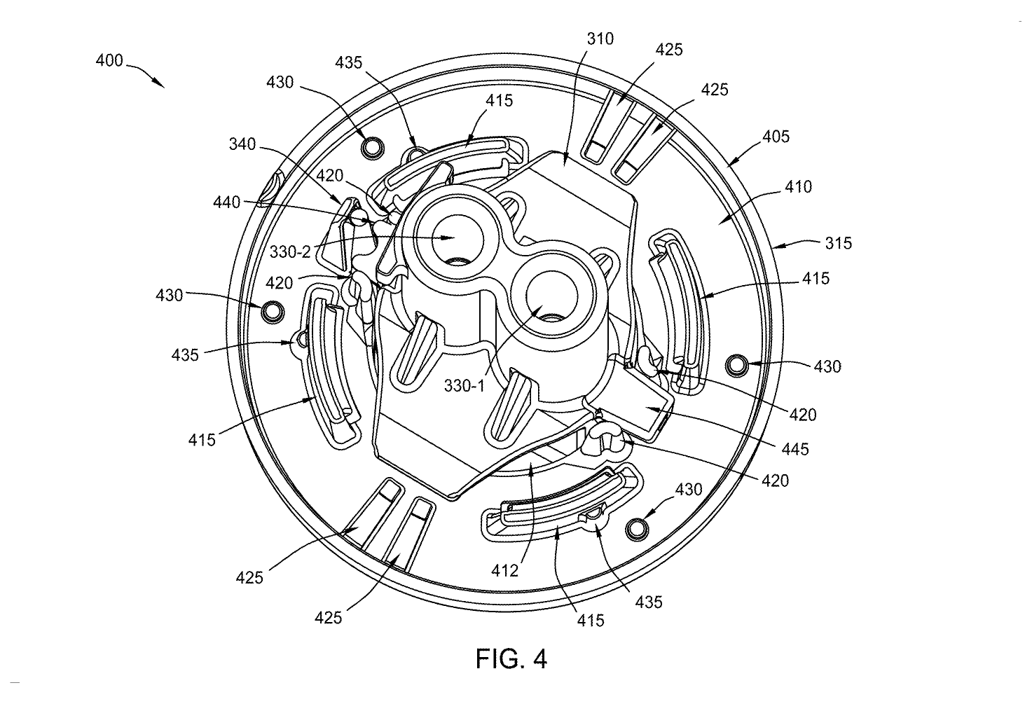

FIG. 4 illustrates a perspective view of a partial assembly 400 of the dual-port connector 305, according to one or more embodiments. The features illustrated in FIG. 4 may be used in conjunction with other embodiments.

The partial assembly 400 comprises the lid member 315 disposed around the core member 310. The base portion 395 of the lid member 315 defines a surface 410 and a rim 405 projecting from the surface 410 along the axial direction. The rim 405 extends as a circumferential ridge around the base portion 395. The surface 410 is shown as an annular surface defining a central opening 412 through which the core member 310 extends (e.g., into and through the annular portion 360 of the lid member 315).

A number of projections extend from the surface 410 for interfacing with various components of the dual-port connector 305. The various projections may also extend along the axial direction and may have height(s) that are less than, equal to, and/or greater than the height of the rim 405. In some embodiments, a plurality of arcuate projections 415 extend from the surface and interface with corresponding portions of the sleeve member 320 to connect the lid member 315 and the sleeve member 320. As shown, the plurality of arcuate projections 415 extend circumferentially around portions of the central opening 412.

In some embodiments, a plurality of axial projections 420 interface with one or more tabs 440, 445 of the core member 310. In some embodiments, the plurality of axial projections 420 are arranged in pairs, such that each of the tabs 440, 445 may be disposed between the axial projections 420 of a respective pair, which limits rotation of the core member 310 relative to the lid member 315. As shown, the plurality of axial projections 420 are disposed at a smaller radius than the plurality of arcuate projections 415 (e.g., closer to the central opening 412). Further, one pair of the axial projections 420 is shown as being radially aligned with the protrusion 340, such that only the shorter tab 440 of the tabs 440, 445 may be fully seated between the pair.

In some embodiments, the plurality of axial projections 420 are frustoconical in shape, and the tabs 440, 445 when fully seated (e.g., contacting the surface 410) are held by an interference fit (or “friction fit”) between the axial projections 420 of the respective pair. The interference fit between the axial projections 420 and the tabs 440, 445 may reduce vibrations and/or rattling between internal components of the dual-port connector 305 when the dual-port connector 305 is connected to a source of vibrations (e.g., surgical console 102) and/or has, for example, pneumatic pulses traveling back and forth through the dual-port connector 305. In some embodiments, the friction fit between the tabs 440, 445 and the axial projections 420 may reduce dead space by filling the inside of the dual-port connector 305, which limits vibration amplitude of the components disposed inside the dual-port connector 305 during exposure to vibrations.

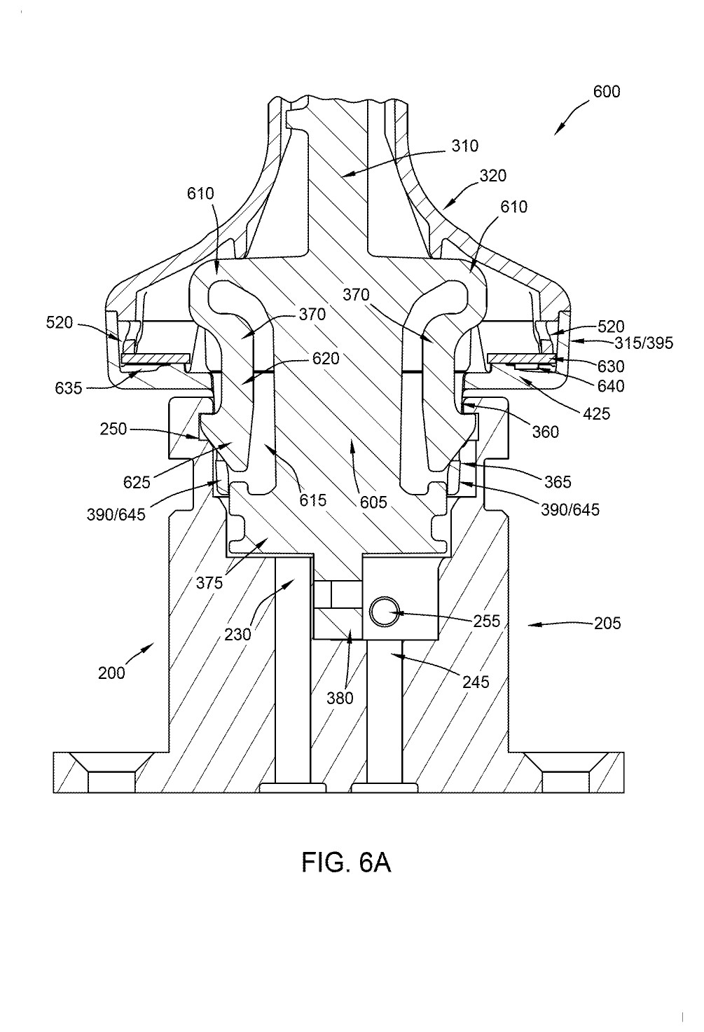

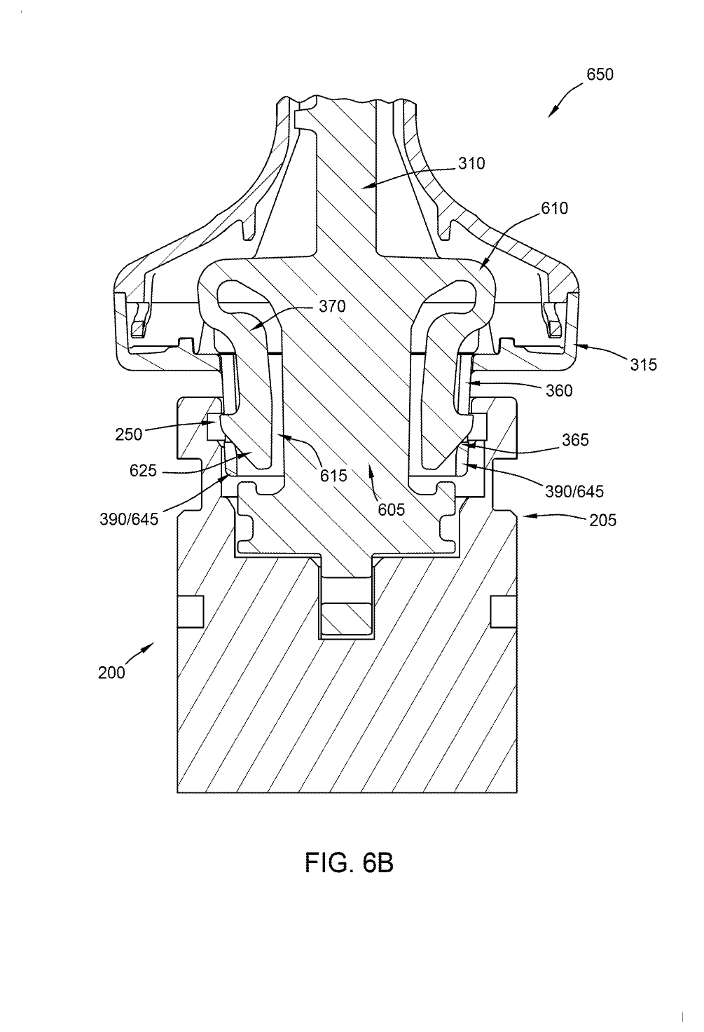

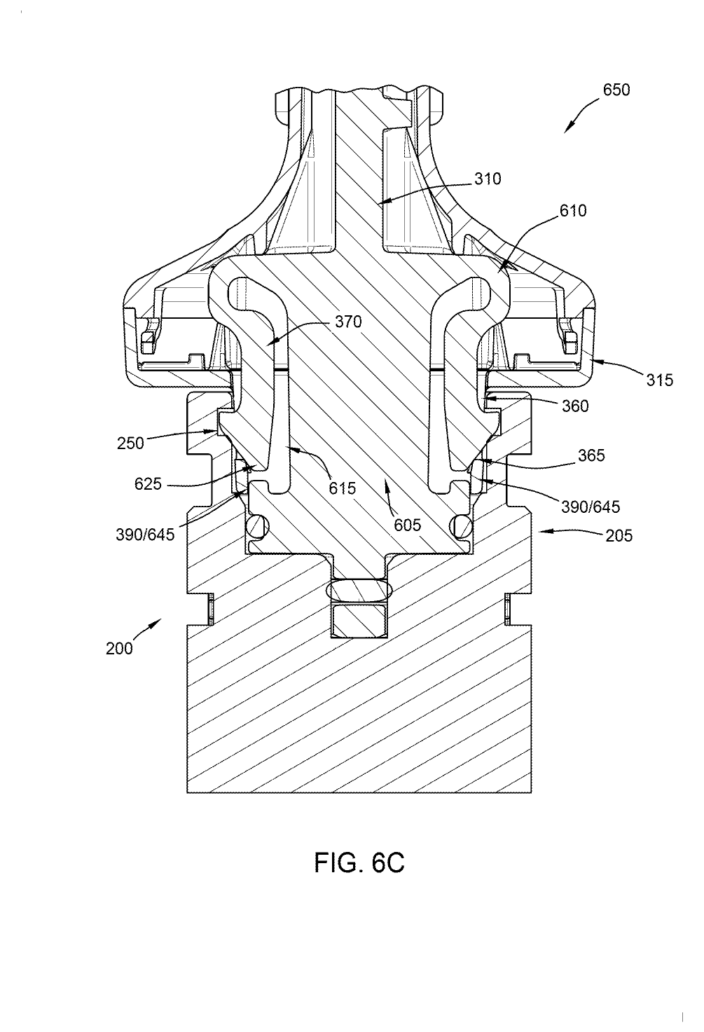

In some embodiments, a plurality of support projections 425, 430 provide a seating surface for a printed circuit board (PCB) of the dual-port connector 305. As shown, the support projections 425 extend radially and have a substantially rectangular profile, and the support projections 430 are disposed circumferentially between the plurality of arcuate projections 415 and the rim 405 (e.g., at a greater radius than the plurality of arcuate projections 415) and have a substantially cylindrical or frustoconical shape. In some embodiments, the support projections 425 include a top surface (e.g., an elevated section) that contacts the PCB, as well as a recessed surface. In this way, an integrated circuit (IC) or other circuitry disposed on the PCB may be arranged in the space between the PCB and the recessed surface. Refer also to FIG. 6A, which includes a PCB 630, a space 635 between the PCB 630 and the support projections 425, and an IC 640 disposed on the PCB 630 and arranged in the space 635. Further, some or all of the arcuate projections 415 may be contoured to include a frustoconical or cylindrical profile 435, such that the PCB is held laterally by an interference fit with the arcuate projections 415.

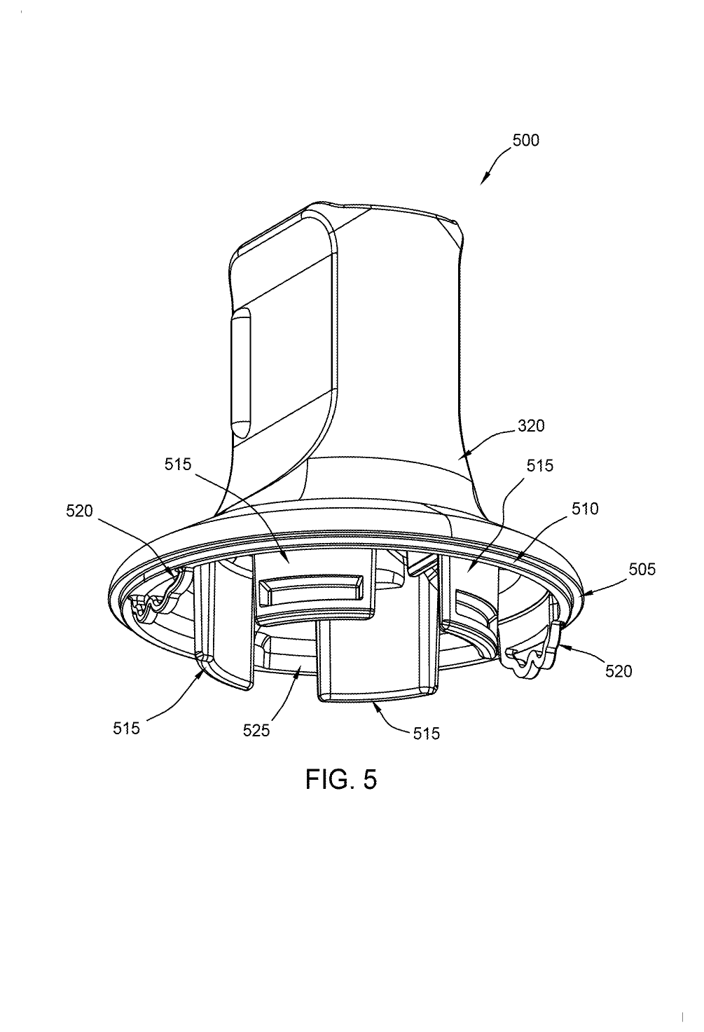

FIG. 5 illustrates a perspective view 500 of a sleeve member 320 of the dual-port connector, according to one or more embodiments. The features illustrated in FIG. 5 may be used in conjunction with other embodiments.

The sleeve member 320 comprises a rim 505 and a circumferential ridge 510 disposed at a smaller radius than the rim 505. The sleeve member 320 further defines a central opening 525 through which the core member 310 extends, and a plurality of arcuate projections 515 that extend circumferentially around portions of the central opening 412. The plurality of arcuate projections 515 extend along the axial direction and interface with the plurality of arcuate projections 415 of the lid member 315 to retain the sleeve member 320 to the lid member 315. For example, the plurality of arcuate projections 515 and the plurality of arcuate projections 415 may include complementary snap-fit features (e.g., spring arms and recesses).

As discussed above, a diameter of the sleeve member 320 at the proximal end may be approximately the same as the diameter of the distal end of the lid member 315. In some embodiments, the rim 505 of the sleeve member 320 aligns with the rim 405 of the lid member 315, and the circumferential ridge 510 laterally contacts the rim 405, whether directly or through an O-ring or other compliant interface.

In some embodiments, the circumferential ridge 510 further defines one or more biasing features 520 that extend proximally and contact the PCB of the lid member 315 when the sleeve member 320 is connected to the lid member 315, helping to retain the PCB in its seated position. The one or more biasing features 520 may be formed of a compliant material and/or may have a compliant geometry. As shown, the one or more biasing features 520 have a rounded “W” shape, but other shapes are also contemplated.

FIG. 6A illustrates a cross-sectional view 600 of the dual-port connector in a retained configuration in the receptacle, according to one or more embodiments. FIG. 6C illustrates an alternate cross-sectional view of the dual-port connector in a retained configuration in the receptacle, according to one or more embodiments. The features illustrated in FIGS. 6A, 6C may be used in conjunction with other embodiments.

In the cross-sectional view 600, the core member 310 comprises a central portion 605 and two (2) spring arms 370 arranged laterally on opposing sides of the central portion 605, although other numbers and arrangements of spring arms are also contemplated. In some embodiments, the spring arms 370 are arranged along a first axis (e.g., a horizontal axis of the page), and the first connector channel 330-1 and the second connector channel 330-2 are arranged on opposing sides of the central portion 605 along a second, orthogonal axis (e.g., an axis extending into and out of the page). In some embodiments, each of the spring arms 370 comprises a shoulder (or “upper arm portion”) 610 contoured to extend laterally from the central portion 605 and transition to a central arm portion 620 that extends substantially along the axial direction (e.g., parallel to a lateral extent of the central portion 605). The central arm portion 620 and the central portion 605 are separated from each other by a space 615. The central arm portion 620 extends to a lower arm portion (or “end portion” or “proximal portion”) 625 that is contoured to extend through the one or more lateral openings 365 of the lid member 315 and engage the internal circumferential channel(s) 250 of the receptacle 140. The shoulder 610 is geometrically designed to be more flexible than other portions of the spring arms 370 (e.g., the central arm portion 620 and the lower arm portion 625), which causes most rotational deformation to occur at the shoulder 610 when enough force is applied.

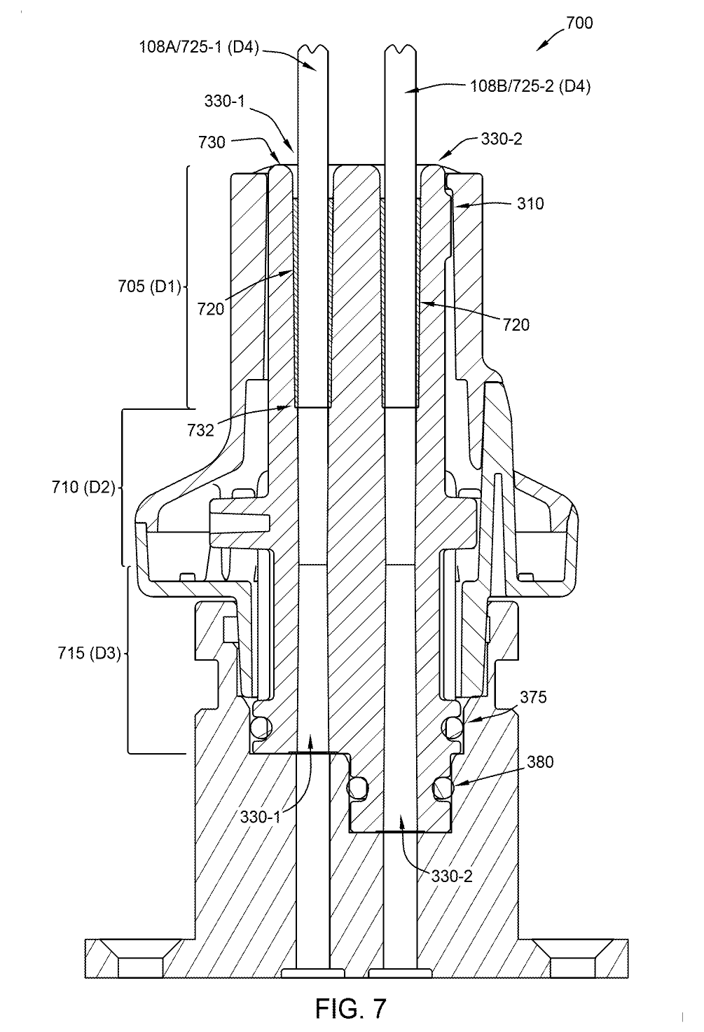

In some embodiments, the shoulders 610 are aligned with the one or more internal circumferential channel(s) 250 along the axial direction. Aligning the shoulders 610 in this manner places a rotationally flexible portion of the spring arms 370 (e.g., the shoulders 610) in line with contact points between the lower arm portion 625 and the internal circumferential channel(s) 250, which may effectively minimize and/or eliminate torque incident on the spring arms 370 responsive to axial forces (e.g., pulling the core member 310 away from the interface component 200). Force resulting from contact between the lower arm portion 625 and the internal circumferential channel(s) 250 has an axial direction aligned (or nearly aligned) with the shoulders 610, which minimizes and/or eliminates torque and/or rotational deformation of the shoulders 610. As such, axial forces caused by positive air pressure in the interface component 200, or caused by a user pulling on the core member 310 (e.g., via tube(s) 725-1 and/or 725-2 shown in FIG. 7), are prevented from causing detachment of the dual-port connector 305 from the interface component 200.

As shown, the lower arm portion 625 includes a sloped edge that contacts a distal end of the receptacle 140 as the annular portion 360 of the dual-port connector 305 is received into the receptacle 140. Contacting the receptacle 140 causes the lower arm portion 625 to deflect medially (e.g., into the space 615, toward the central portion 605) as the lower arm portion 625 contacts a sidewall of the receptacle 140 (e.g., partly defining the recess 220). As the annular portion 360 is received further into the receptacle 140, the lower arm portion 625 reaches the internal circumferential channel(s) 250 and no longer contacts the sidewall of the receptacle 140. The lower arm portion 625 displaces laterally and is received into the internal circumferential channel(s) 250. When so received, the lower arm portion 625 may return to its neutral configuration or may have a medial deflection that is less than that when contacting the sidewall. The lower arm portion 625 further includes a flat back that contacts the internal circumferential channel(s) 250 and prevents the lower arm portion 625 from releasing from the internal circumferential channel(s) 250, such that the dual-port connector 305 is retained in the receptacle 140.

The lid member 315 further comprises one or more release features 390 that depress the one or more spring arms 370 to release the dual-port connector 305 from the receptacle 140. In some embodiments, the one or more release features 390 comprise proximal portions 645 of the annular portion 360. FIG. 6B illustrates a cross-sectional view 650 of the dual-port connector 305 when releasing from the receptacle, according to one or more embodiments.

In some embodiments, an axial pulling force on the lid member 315 (or on the sleeve member 320 attached thereto) causes the annular portion 360 to slide in a distal direction (as shown, an upward direction), which causes the proximal portions 645 to contact the sloped edge of the lower arm portion 625 and apply a medial force to the lower arm portion 625. Once the lower arm portion 625 is depressed into the space 615 to withdraw the lower arm portion 625 from the internal circumferential channel(s) 250, the dual-port connector 305 is released from the receptacle 140 and may be removed from the receptacle 140 by the axial pulling force.

In some embodiments, the dual-port connector 305 is configured such that applying an axial pulling force on the core member 310 (e.g., pulling on the attached tubing) does not release the dual-port connector 305 from the receptacle 140. The tubing may be attached to the first connector channel 330-1 and the second connector channel 330-2 using any suitable techniques. For example, FIG. 7 illustrates a cross-sectional view 700 of the core member 310 having a first tube and a second tube retained therein, according to one or more embodiments. The features illustrated in FIG. 7 may be used in conjunction with other embodiments.

In the cross-sectional view 700, a first tube 725-1 and a second tube 725-2 (representing examples of the connection conduits 108A and 108B) are received into the first connector channel 330-1 and the second connector channel 330-2, respectively. Each of the first connector channel 330-1 and the second connector channel 330-2 defines a respective distal portion 705 having an inner diameter D1, a mid-portion 710 having an inner diameter D2, and a proximal portion 715 having an inner diameter D3. The inner diameter D1 monotonically decreases from a distal end 730 of the distal portion 705 to a proximal end 732 of the distal portion 705. The inner diameters D2 and D3 are less than or equal to the inner diameter D1 at the proximal end 732 of the distal portion 705. The inner diameter D2 is equal to the inner diameter D3, such that the mid-portion 710 and the proximal portion 715 are combined into a straight bore. However, in some embodiments, the inner diameter D3 may be less than the inner diameter D2. In some embodiments, the inner diameters D2 and D3 may be equal at the location where the mid-portion 710 and the proximal portion 715 come into contact, but increase in size as distance from the contact location increases.

At the distal end 730 of the distal portion 705, the inner diameter D1 is greater than an outer diameter D4 of the corresponding one of the first tube 725-1 and the second tube 725-2 to allow for insertion of the first tube 725-1 and the second tube 725-2 into the distal portion 705. However, at the proximal end 732 of the distal portion 705, the inner diameter D1 is less than or equal to the outer diameter D4 such that the first tube 725-1 and the second tube 725-2 are retained in the distal portion 705 (e.g., at the proximal end 732) by an interference fit. Further, the inner diameter D2 of the mid-portion 710 is less than or equal to the outer diameter D4, such that the first tube 725-1 and the second tube 725-2 do not slide past the proximal end 732.

In some embodiments, an adhesive 720 is applied into the first connector channel 330-1 and the second connector channel 330-2 outside the respective one of the first tube 725-1 and the second tube 725-2 to retain the tubes in the first connector channel 330-1 and the second connector channel 330-2. One non-limiting example of the adhesive 720 include solvent bonding, where a solvent is applied to exterior surfaces of the first tube 725-1 and the second tube 725-2, and the components bond with the first connector channel 330-1 and the second connector channel 330-2 when pressed together. Another non-limiting example of the adhesive 720 is an epoxy that is cured by ultraviolet (UV) light. By controlling the inner diameters D1, D2, D3 and the lengths of the distal portion 705, the mid-portion 710, and the proximal portion 715, the amount and location of the applied adhesive 720 may be more precisely controlled, which can improve the manufacturing process and ensure the core member 310 is capable of performing as designed. For example, the inner diameter D2 may be equal to the outer diameter D4 to minimize any ingress of the adhesive 720 into the mid-portion 710, and the length of the distal portion 705 may be selected to allow a predetermined volume of adhesive 720 to be applied in the distal portion 705 without leaking out a distal end of the first connector channel 330-1 and the second connector channel 330-2.

While the foregoing is directed to embodiments of the present disclosure, other and further embodiments of the disclosure may be devised without departing from the basic scope thereof, and the scope thereof is determined by the claims that follow.

What is claimed is:

1. A dual-port connector configured to connect to a receptacle of a surgical console, the dual-port connector comprising:
a core member comprising:
a first connector channel configured to fluidly connect a first receptacle channel of the receptacle with a first tube connected to a surgical instrument;
a second connector channel configured to fluidly connect a second receptacle channel of the receptacle with a second tube connected to the surgical instrument; and
one or more spring arms configured to retain the dual-port connector in the receptacle; and
a lid member disposed around the core member, the lid member comprising:
one or more lateral openings through which a portion of the one or spring arms extends to connect the core member to the receptacle; and
one or more release features configured to depress the one or more spring arms to release the dual-port connector from the receptacle.

2. The dual-port connector of claim 1, further comprising:
a sleeve member connected to the lid member, wherein the lid member is configured to, responsive to an axial pulling force applied to the sleeve member, slide along the core member to cause the one or more release features to depress the one or more spring arms.

3. The dual-port connector of claim 2,
wherein the first connector channel is configured to receive the first tube therein,
wherein the second connector channel is configured to receive the second tube therein, and
wherein the dual-port connector is configured to be retained in the receptacle responsive to an axial pulling force applied to the core member.

4. The dual-port connector of claim 3,
wherein each of the first connector channel and the second connector channel comprises a respective distal portion having:
a distal end where an inner diameter of the respective distal portion is greater than an outer diameter of a corresponding one of the first tube and the second tube, and
a proximal end where the inner diameter of the respective distal portion is less than or equal to the outer diameter of the corresponding one of the first tube and the second tube, and
wherein the first tube and the second tube are configured to be retained in the first connector channel and the second connector channel, respectively, by applying an adhesive into the respective connector channel outside the respective tube.

5. The dual-port connector of claim 1,
wherein the receptacle comprises one or more internal circumferential channels configured to receive the portion of the one or more spring arms, and
wherein each of the one or more spring arms comprises a shoulder aligned with the one or more internal circumferential channels, a central arm portion, and a lower arm portion that extends through the one or more lateral openings.

6. The dual-port connector of claim 1,
wherein the first connector channel extends to a surface of the core member to define a first proximal male coupling that is configured to be received at a distal end of the first receptacle channel.

7. The dual-port connector of claim 6,
wherein the second connector channel extends through the surface to define a second proximal male coupling that is configured to be received at a distal end of the second receptacle channel.

8. The dual-port connector of claim 1, further comprising:
a plurality of axial projections on the lid member; and
one or more tabs on the core member,
wherein the plurality of axial projections on the lid member are configured to interface with the one or more tabs on the core member to reduce vibration amplitude within the dual-port connector.

9. The dual-port connector of claim 1, further comprising:
a printed circuit board (PCB) coupled to the lid member; and
a sleeve member connected to the lid member,
wherein a first side of the PCB contacts one or more projections of the lid member, and
wherein a biasing feature of the sleeve member contacts a second, opposing side of the PCB.

10. A surgical system comprising:
a surgical console having a receptacle comprising a first receptacle channel, a second receptacle channel, and one or more recesses;
a surgical instrument fluidly connected with a first tube and a second tube; and
a snap-fit connector fluidly connecting the first tube and the second tube to the first receptacle channel and the second receptacle channel, respectively, the snap-fit connector comprising:
a core connected with the first tube and the second tube, the core comprising one or more spring arms; and
a lid disposed around the core, wherein the one or more spring arms extend through one or more lateral openings of the lid into the one or more recesses to retain the snap-fit connector in the receptacle, and wherein the lid comprises one or more release features configured to, responsive to an axial pulling force applied to the lid, depress the one or more spring arms to release them from the one or more recesses.

11. The surgical system of claim 10, the snap-fit connector further comprising:
a sleeve connected to the lid,
wherein the lid is configured to, responsive to an axial pulling force applied to the sleeve, slide along the core to cause the one or more release features to depress the one or more spring arms.

12. The surgical system of claim 11, wherein the core comprises:
a first connector channel configured to receive the first tube therein; and
a second connector channel configured to receive the second tube therein, and
wherein the snap-fit connector is retained in the receptacle responsive to an axial pulling force applied to the core.

13. The surgical system of claim 12, wherein each of the first connector channel and the second connector channel comprises a respective distal portion having:
a distal end where an inner diameter of the respective distal portion is greater than an outer diameter of a corresponding one of the first tube and the second tube, and
a proximal end where the inner diameter of the respective distal portion is less than or equal to the outer diameter of the corresponding one of the first tube and the second tube, and
wherein the first tube and the second tube are configured to be retained in the first connector channel and the second connector channel, respectively, by applying an adhesive into the respective connector channel outside the respective tube.

14. The surgical system of claim 10, wherein the surgical console comprises:
a sensor configured to detect a presence of the core when the snap-fit connector is retained in the receptacle; and
one or more processors configured to initiate, responsive to the sensor detecting the presence of the core, wireless communication with circuitry of the snap-fit connector.

15. The surgical system of claim 14, wherein the one or more processors are further configured to:
receive, from the circuitry of the snap-fit connector, identification information that identifies one or both of the snap-fit connector and the surgical instrument; and
configure operation of the surgical console responsive to the identification information.
